(19) United States
(12) Patent Application Publication
(10) Pub. No.: US 2026/0002873 A1
(43) Pub. Date: Jan. 1, 2026

(54) OPTICAL NANOSENSORS FOR HYDROLYTIC ENZYME ACTIVITY ON SOLID SUBSTRATES

(71) Applicant: Iowa State University Research Foundation, Inc., Ames, IA (US)

(72) Inventors: Nigel Forest Reuel, Ames, IA (US); Abbas Elhambakhsh, Ames, IA (US)

(21) Appl. No.: 19/235,010

(22) Filed: Jun. 11, 2025

Related U.S. Application Data
(60) Provisional application No. 63/664,490, filed on Jun. 26, 2024.

(51) Int. Cl. G01N 21/64; C12Q 1/26; C12Q 1/37; C12Q 1/40; G01N 21/77; G01N 27/327

(52) U.S. Cl. CPC G01N 21/6428; C12Q 1/26; C12Q 1/37; C12Q 1/40; G01N 27/3278; G01N 2021/6432; G01N 2021/7786; G01N 2333/90232; G01N 2333/942; G01N 2333/948

(57) ABSTRACT

A sensor assembly probe may include a fluorescent semi-conductive nanoparticle. A sensor assembly probe may include a hydrophobic substrate for a predetermined enzyme, associated with the fluorescent semi-conductive nanoparticle by a non-covalent electrostatic interaction. A sensor assembly probe may include a hydrophilic group bonded to the hydrophobic substrate.

### CROSS-REFERENCE TO RELATED APPLICATION

This application claims the benefit of priority to U.S. Provisional Patent Application Ser. No. 63/664,490 entitled “OPTICAL NANOSENSORS FOR HYDROLYTIC ENZYME ACTIVITY ON SOLID SUBSTRATES,” filed Jun. 26, 2024, the disclosure of which is incorporated herein in its entirety by reference.

### STATEMENT OF GOVERNMENT SUPPORT

This invention was made with Government support under Award No. OIA2242763 awarded by NSF. The U.S. Government has certain rights in this invention.

### BACKGROUND

Determining the presence and activity of an enzyme can be useful in many different contexts. In some applications, this can be done indirectly by detecting the presence of byproducts of the reaction between an enzyme and substrate. Indirect detection can be unreliable and potentially expensive. It may, therefore, be desirable to develop improved detection methods and assemblies.

### SUMMARY OF THE DISCLOSURE

In some aspects, the techniques described herein relate to a sensor assembly probe for determining enzymatic activity, the sensor assembly probe including: a fluorescent semi-conductive nanoparticle; a hydrophobic substrate for a predetermined enzyme, associated with the fluorescent semi-conductive nanoparticle by a non-covalent electrostatic interaction; and a hydrophilic group bonded to the hydrophobic substrate.

### BRIEF DESCRIPTION OF THE FIGURES

The drawings illustrate generally, by way of example, but not by way of limitation, various embodiments discussed in the present document.

FIG. 1 is a schematic depiction of mechochemical formation of ion pairs between lysine and lignin.

FIG. 2A is a graph showing the response of LacAS at 40 ppm with controls of heat denatured enzymes.

FIG. 2B is a graph showing the response of LacTV at 40 ppm with controls of heat denatured enzymes.

FIG. 2C shows enzyme concentration dependence of signal change for LLS to LacAS showing LOD of 0.25 ppm.

FIG. 3 is a schematic diagram showing three zones we sampled soil from for method application: (A) Rhizosphere, (B) Row, and (C) Interrow.

FIG. 4A is a graph showing the response of LLS to available enzymes in the rhizosphere soil sample.

FIG. 4B is a graph showing the response of LLS to available enzymes in the row soil sample.

FIG. 4C is a graph showing the response of LLS to available enzymes in the rhizosphere interrow soil sample.

FIG. 5A is a graph showing the response of LLS to enzyme available in rhizosphere soil samples after two weeks and under preservation temperatures of −20° C.

FIG. 5B is a graph showing the response of LLS to enzyme available in rhizosphere soil samples after two weeks and under preservation temperatures of 4° C.

FIG. 5C is a graph showing the response of LLS to enzyme available in rhizosphere soil samples after two weeks and under preservation temperatures of 25° C.

FIG. 5D is a graph showing the response of LLS to enzyme available in rhizosphere soil samples after four weeks and under preservation temperatures of −20° C.

FIG. 5E is a graph showing the response of LLS to enzyme available in rhizosphere soil samples after four weeks and under preservation temperatures of 4° C.

FIG. 5F is a graph showing the response of LLS to enzyme available in rhizosphere soil samples after four weeks and under preservation temperatures of 25° C.

FIG. 6A is a graph showing the response of LLS to the enzymes available in Sparta soil types.

FIG. 6B is a graph showing the response of LLS to the enzymes available in Coland soil types.

FIG. 6C is a graph showing the response of LLS to the enzymes available in Nicollet soil types.

FIG. 7 is a graph showing the response of Zein-Lysine-SWCNT to bacterial protease.

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The term “weight-average molecular weight” as used herein refers to M_{w}, which is equal to ΣMᵢ²nᵢ/ΣMᵢnᵢ, where nᵢ is the number of molecules of molecular weight Mᵢ. In various examples, the weight-average molecular weight can be determined using light scattering, small angle neutron scattering, X-ray scattering, and sedimentation velocity.

Hydrolytic enzyme screening has been found to be a vital step for varying industrial and natural processes. In nature, hydrolytic enzymes like ligninases, proteases, cellulases, etc., are also responsible for converting organic compounds into crucial nutrients required for plant growth including phosphorus, sulfur and nitrogen in mineralized states. The detection of these enzymes is a powerful tool for evaluating the level of soil health as well as introducing a technique to anticipate the available and required nutrients in farmland soils. Screening enzymatic degradation through the functional single-wall carbon nanotubes (SWCNTs) has gained much attention for sensing enzyme activities in recent studies. SWCNTs shows unique fluorescence properties, emitting in the near-infrared region (nIR), which makes them a perfect candidate for biologically relevant purposes due to water's reduced absorption in this range. The sensitivity of SWCNTs is notably high, allowing for distinct fluorescence signals to be observed for a SWCNT in its bare state, after substrate-wrapping, and upon contact with the targeted enzymes. This difference in the fluorescence signals can offer a wide range opportunities for sensor applications.

However, due to the necessity of conducting enzyme activity screening in aqueous media, the utilization of SWCNT-based probes is limited to hydrophilic elements or derivatives of elements that enable SWCNTs to become soluble in liquid media. As a result, the use of varying hydrophobic elements has stayed a challenge to detect several enzyme activities. In addition, the use of the derivatives of elements is another obstacle which may affect the reliability of the obtained data.

An approach for these problems can be the coupling of hydrophobic elements to an appropriate hydrophilic agent before non-covalently functionalization on SWCNTs through mechanochemistry approach. Mechanochemical techniques have been recently presented for varying chemical coupling processes in materials synthesis and organic chemistry. Their high flexibility has also resulted in some success in creating metal-organic frameworks (MOFs), coordination polymers and distinctive pharmaceutical cocrystals. Mechanochemical methods have a negligible environmental footprint and can lead to highly efficient and selective processes, making them ideal candidates for surface modification of nanoparticles and coupling processes. Their straightforward procedures also enable the use of a variety of precursors, resulting in minimal waste generation during the process.

This disclosure is directed towards various examples of a sensor assembly probe and facile methods of making the sensor assembly probe. The sensor assembly probe can be used for determining enzymatic activity. Specifically, the sensor assembly probe can be used to determine enzymatic activity for an enzyme used to degrade a hydrophobic substrate.

Many enzymes that would be of interest for detection, can have hydrophobic substrates. However, getting a sensor assembly having a hydrophobic substrate in solution can be challenging. The instant disclosure provides a sensor assembly that allows for a hydrophobic substrate to be quicky and easily included.

In operation, the nanoparticles have a detectible fluorescent emission when in solution and the hydrophobic substrate is bonded thereto.

According to one non-limiting theory, upon contact with the predetermined enzyme, the hydrophobic substrate is degraded. Degradation can occur by bonds in the hydrophobic substrate being hydrolyzed or otherwise cleaved, for example, by removing a charged group. This results in the nanoparticles being released and adhering to each other. This quenches or at least decreases the fluorescent signal of the nanoparticles. The change in fluorescent signal indicates that the predetermined enzyme of interest is present in solution. According to another non-limiting theory, the degradation can result in the release of the nanoparticles which can limit access to the solvent present and a quenched signal.

In some examples, instead of signal quenching or reduction being indicative of degradation, an increase in signal intensity can be indicative of an enzyme of interest being present. For example, it is possible that the solution to which the nanoparticles are disposed in, can include brightening agents such as a surfactants of small organic molecules capable of adhering to the surface of the nanoparticles both of which can help to prevent or mitigate aggregation of the nanoparticles such that the fluorescent signal is not quenched.

The nanoparticles can include any suitable material. Examples of suitable materials can include a ceramic material (e.g., aluminum oxide or copper (II) oxide), a polymer, a glass-ceramic, a composite, a metal carbide (e.g., SiC), a nitride (e.g., aluminum nitride, silicon nitride), a metal (e.g., Al, Cu, Au, Ag), a non-metal (e.g., graphite and carbon). The nanoparticle can have any suitable morphology. For example, the morphology of the nanoparticle can be chosen from a nanosphere, a nanorod, a nanofiber, a nanotube, a nanostar, a nanocup, or combinations thereof. At least one of a length, width, and diameter of the nanoparticle is in a range of from about 0.5 nm to about 10,000 nm, about 1 nm to about 100 nm, about 10 nm to about 50 nm, about 100 nm to about 2,500 nm, about 2,500 nm to about 10,000 nm, or less than, equal to, or greater than about 0.5 nm, 0.7, 1, 25, 50, 100, 500, 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, or about 10,000 nm. Generally, nanoparticles in which at least one of a length, width, and diameter of the nanoparticle is in a range of from about 0.5 nm to about 100 nm are classified as ultrafine nanoparticles. Generally, nanoparticles in which at least one of a length, width, and diameter of the nanoparticle is in a range of from about 100 nm to about 2,500 nm are classified as fine nanoparticles. Generally, nanoparticles in which at least one of a length, width, and diameter of the nanoparticle is in a range of from about 2,500 nm to about 10,000 nm are classified as coarse nanoparticles. The morphology of the nanoparticles can be uniform.

The sensor assembly probe can include a plurality of the nanoparticles. Respective individual nanoparticles can have at least one of substantially the same morphology, substantially the same dimensions, and have substantially the same composition. Alternatively, the respective individual nanoparticles can differ in at least one of their morphologies, dimensions, and compositions. The plurality of nanoparticles can be heterogeneously or homogenously distributed in the aqueous medium.

When at least one nanoparticle is contacted with the hydrophobic substrate and in solution, the nanoparticle fluoresces. In some examples, the nanoparticle can fluoresce at wavelengths ranging from about 800 nm to about 1500 nm, 950 nm to about 1100 nm, or less than, equal to, or greater than about 800, 810, 820, 830, 840, 850, 860, 870, 880, 890, 900, 910, 920, 930, 940, 950, 960, 970, 980, 990, 1000, 1100, 1200, 1300, 1400, or about 1500 nm. In examples where the sensor assembly probe includes a plurality of nanoparticles, respective nanoparticles can fluoresce at substantially the same frequency.

In other examples where the sensor assembly includes a plurality of nanoparticles, respective nanoparticles can fluoresce at different frequencies. For example, the respective fluorescent signals emitted by the first fluorescent semi-conductive nanoparticle and the second fluorescent semi-conductive nanoparticle have intensities of fluorescence that differ by about 0 to 100%, about 0 to 20% or less than, equal to, or greater than about 0%, 5, 10, 15, 20, 25, 30, 35, 40, 45, 50, 55, 60, 65, 70, 75, 80, 85, 90, 95, or 100%.

The hydrophobic substrate for the predetermined enzyme can be selected from many suitable candidates. In some examples, the solid-state substrate can include a bond that is hydrolysable or oxidizable by the predetermined enzyme. Examples of such bonds can include an ester bond, a glycosylic bond, an ether bond, a peptide bond, an acid anhydride bond, a halide bond, a phosphorous-sulfur bond, a sulfur-sulfur bond, a carbon-phosphorous bond, a carbon-sulphur bond, or a combination thereof. The hydrophobic substrate can include lignin, a polysaccharide, a protein, or a lipid or a combination thereof.

The hydrophobic substrate is bonded to the fluorescent semi-conductive nanoparticle through a non-covalent electrostatic interaction. The hydrophilic group is bonded to the hydrophobic substrate. The hydrophilic group includes a functional group that is capable of solubilizing the hydrophobic substrate and therefore the sensor probe assembly.

The predetermined enzyme in the sensor probe assembly can be any suitable enzyme. The enzyme is selected to react with the hydrophobic substrate. The sensor assembly probe can include more than one types of enzymes. In examples that include more than one enzyme, the enzymes can be the same enzyme or a mixture of different enzymes. Where different enzymes are present in the assembly, the different enzymes can be adapted to react with different hydrophobic substrates. The hydrophobic substrate that a particular enzyme reacts with may be present in the assembly or may not be present in the assembly.

The predetermined enzyme or enzymes may belong to any class of enzymes. For example, the enzyme or enzymes may be classified as a hydrolase (alternatively known as an EC 3 enzyme). The hydrolase can be classified by the bond it acts upon. For example, the hydrolase can be chosen from a phytase, an esterase, nuclease, phosphodiesterase, lipase, phosphatase, DNA glycosylase, glycoside hydrolase, proteases, peptidase, acid anhydride hydrolase, helicase, GTPase, or mixtures thereof. A cellulase can also be the predetermined enzyme. Examples of a protease can include a cysteineprotease, a serineprotease, a threonineprotease, an aspartic protease, a glutamic protease, a metalloprotease, a PA clan protease, or a mixture thereof. Examples of a cellulose can include endo-1,4-beta-D-glucanase (beta-1,4-glucanase, beta-1,4-endoglucan hydrolase, endoglucanase D, 1,4-(1,3,1,4)-beta-D-glucan 4-glucanohydrolase), carboxymethyl cellulase (CMCase), avicelase, celludextrinase, cellulase A, cellulosin AP, alkali cellulase, cellulase A 3, 9.5 cellulase, and pancellase SS. Examples of oxidases can include glucose oxidase, monoamine oxidase, cytochrome p450 oxidase, NADPH oxidase, xanthine oxidase, L-gulonolactone oxidase, laccase, lysyl oxidase, polyphenol oxidase, sulfhydryl oxidase, or a mixture thereof.

The sensor assembly can be used according to any suitable method. According to various examples, the method can include dispensing the sensor assembly probe in solution. The solution is typically aqueous. When the nanoparticles are in solution, they produce a fluorescent emission. The initial fluorescence is measured. In some examples where the assembly includes a mixture of nanoparticles that produce different fluorescent emissions, multiple emissions may be measured

The enzyme or mixture of enzymes are then contacted with the nanoparticles and hydrophobic substrates. If an enzyme is associated with a particular hydrophobic substrate, the hydrophobic substrate will be degraded and release the nanoparticles. When the nanoparticles are released, their fluorescence can decrease or disappear. The fluorescent signal then disappears as a result of the nanoparticles agglomerating and their signal being quenched. Thus, a measured second fluorescent emission will have a different intensity than the first fluorescent emission or there will be no fluorescent emission and will be indicative of the substrate being degraded.

Measuring a second fluorescent emission that is different than the first fluorescent emission confirms the presence of a predetermined enzyme. In examples where nanoparticles having different fluorescent emissions and different substrates attached thereto are present, a decrease in the emission in one or both of the nanoparticles can indicate the presence of two different predetermined enzymes. In this manner, the presence of one or more enzymes in a mixture of enzymes or another constituent of a solution can be confirmed. Additionally, the rate of reaction between the predetermined enzyme and a substrate analogue can be determined by monitoring the rate at which the fluorescent emission intensity changes.

The sensor assembly probe described herein can be formed relatively easily and reliably using sonication. Sonication is not typically used to form chemical bonds directly. Sonication, is a process that uses sound waves, usually ultrasonic frequencies, to agitate particles in a sample. This agitation can promote various chemical and physical processes. For example. Sonication can increase the reaction rate of a chemical process. The ultrasonic waves can cause intense mixing and produce localized high temperatures and pressures (through phenomena like cavitation). This can make reactants come into contact more frequently and energetically, potentially overcoming activation energy barriers more efficiently.

To form the sensor probe assembly, the hydrophobic substrate and hydrophilic group are contacted with each other under conditions sufficient to product a reaction. The mixture is sonicated to enhance the reaction kinetics as described hereinabove. Any excess (e.g., unreacted or non-bonded) hydrophilic groups are washed away. The bonded hydrophobic substrate and hydrophilic group are then contacted with the semi-conductive nanoparticle. That mixture is then sonicated to facility bonding between the hydrophobic substrate and the and the semi-conductive nanoparticle. Any sonication step can range from about 5 minutes to about 60 minutes, about 15 minutes to about 25 minutes, less than, equal to, or greater than about 5 minutes, 10, 15, 20, 25, 30, 35, 40, 45, 50, 55, or about 60 minutes.

### Examples

Various examples of the present disclosure can be better understood by reference to the following Examples which are offered by way of illustration. The present disclosure is not limited to the Examples given herein.

### Chemistry and Material Preparation

Lignin is known to possess hydroxyl (2-8%, w/w) and carboxylic acid (1-8%, w/w) functional groups. Sonication of SWCNT with either lysine or lignin alone lead to unstable dispersions with pronounced sedimentation, as the lignin is too hydrophobic alone to stay in suspension and the lysine is not hydrophobic enough to electrostatically associate with the SWCNT. Sonication of lignin and lysine together prior to sonication with SWCNT, however, led to a stable solution even after prolonged centrifugation. This result indicated mechanochemical conjugation between the lignin and lysine in the first sonication step, creating a wrapping capable of wrapping the SWCNT surface but remaining charged enough to stay in suspension. Indeed, after extensive dialysis of the lignin-lysine material to remove free lysine, elemental analysis revealed a 10-fold increase in the weight percentage of nitrogen in the material. It is thought that either a covalent bond or ion pairing was forming between the lignin and lysine under the conditions of sonication to create a stable linkage. This hypothesis was further supported by the observation of a characteristic C═O peak (1637 cm-1) in the IR spectrum of the lignin-lysine conjugate that was not present in either lignin or lysine starting materials.

To determine if the mechanochemical coupling was either ionic pairing or covalent bond formation to further analysis of a simpler molecule was conducted. Direct characterization of the lignin-lysine conjugate via NMR was not possible because the size of the lignin particles leading to slow tumbling on the NMR time scale and loss of signal. Instead, anthracene was selected as a proxy insoluble substrate as it is hydrophobic with demonstrated association to SWCNT and commercially available with either a single carboxylic acid or a hydroxyl side group, 9-Anthracenecarboxylic acid (9A-COOH) and 9-Anthracenemethanol (9A-OH) respectively; these reagents were used to assess if the mechanochemically induced bond is more likely between the more reactive carboxylic acid groups than the hydroxyl groups both present in lignin and to determine the nature of the mechanochemically induced coupling. After tip sonication with lysine, 9A-OH crashed out of solution (no coupling) and 9A-COOH presented a stable and homogenized solution. The lysine-9A-COOH composite was then employed to create water-dispersible 9A-Lysine-SWCNT. The solubility of bare 9A-COOH and 9A-COOH-lysine in water was compared, and as can be seen, 9A-COOH-lysine is significantly more water-soluble compared to 9A-COOH alone, supporting our hypothesis about the N—COOH coupling process.

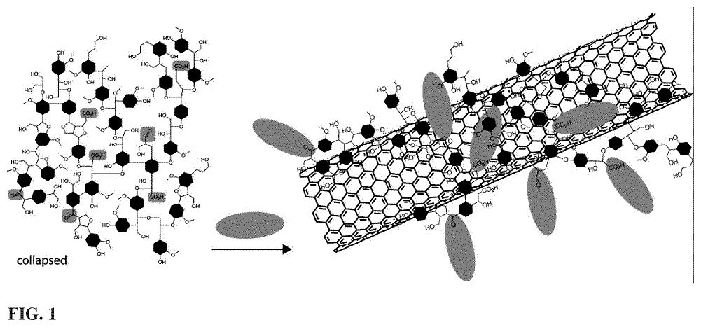

LCMS analysis of 9A-COOH-Lysine-SWCNT did not show evidence of covalent bond formation, therefore we posit that most of this mechanochemical coupling is induced ion pairing. In the case of lignin, the input energy exfoliates the substrate and introduces opportunities for proton transfer between the carboxylic acids of lignin and the amines of lysine. LCMS data was obtained on 9A-acetaldehyde sonicated with lysine, which suggests formation of an imine between lysine and the aldehyde groups of anthracene. However, such bonds would not be hydrolytically stable and we do not see solubility stability of the 9A-acetaldehyde-Lysine SWCNT. The mechanochemical functionalization is summarized in the schematic diagram of FIG. 1.

Observations after Mechanochemical Coupling

To visualize probe stability after mechanochemical coupling, the three aqueous solutions of lysine-SWCNT (without lignin), Lignin-SWCNT (without lysine), and LLS after 30 minutes of centrifugation at 10,000 rpm (11,180 g) were visualized. As observed, lysine-SWCNT and Lignin-SWCNT dispersions are unstable and crash out of solution, whereas no sedimentation was observed at the bottom of the tube for the LLS solution after prolonged centrifugation, indicating the success of the coupling and solubilization process.

### Selectivity and Sensitivity.

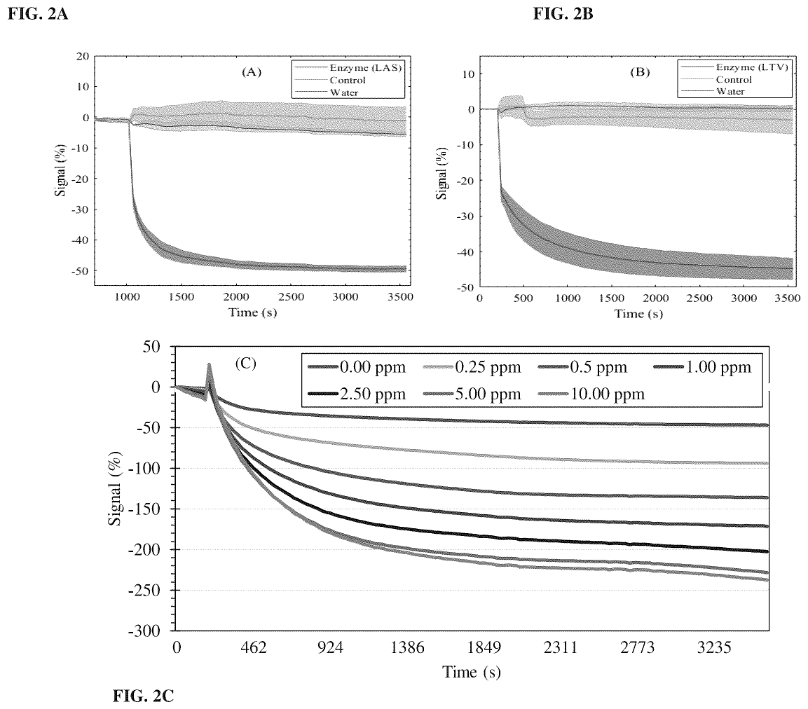

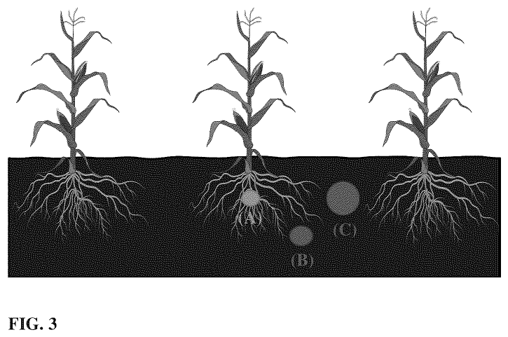

The sensitivity of LLS to hydrolytic degradation was checked by two well-known ligninase Laccase from Aspergillus species (LacAS, SAE0050, Sigma) and Laccase from Trametes versicolor (LacTV, 38429, Sigma). Nanoprobe work with amphiphilic wrappings shows that upon enzyme addition there was a strong decrease in signal attributed to hydrolases attacking the substrate coating, increasing solvent access, and thereby quenching signal response. A similar response is observed for LLS to both types of ligninases (FIG. 2A-B). However, its sensitivity was greater for LacAS, and the lignin element on SWCNT was degraded in a shorter time period (900 s after adding the ligninase solution, the signal line for LLS became relatively constant), compared to LacTV, where degradation continued even after 3500 s. FIG. 3C also illustrates the response of LLS to LacAS within the concentration range of 0 to 10 ppm, showcasing not only the sensitivity of LLS but also its capability to quantify the proportion of ligninase compounds at low concentrations (less than 0.5 ppm).

### Application of Method for Measuring Potential Soil Hydrolase Activity

Assessment of potential soil enzymatic activity was chosen as an application of this new method to demonstrate sensitivity and utility of these probes. Soil samples were using the “shovel-and-shake” method to collect soil from three zones: (A) the rhizosphere (soil closely adhering to the plant's roots), (B) the row (under the plant roots but not directly attached), and (C) the interrow (furthest distance from the plant's roots) (FIG. 3). Soil was sampled through these three zones from a field of Zea mays (maize) that was under consistent management for nearly 15 years. According to the literature on potential enzyme activity in soil, hydrolytic enzyme activity is expected to increase the closer to the plant root, from (C) to (A) because of greater microbial biomass and greater influence of maize roots.

Consequently, it is anticipated that higher enzyme activity would be observed in the rhizosphere and lower enzyme activity in the interrow areas.

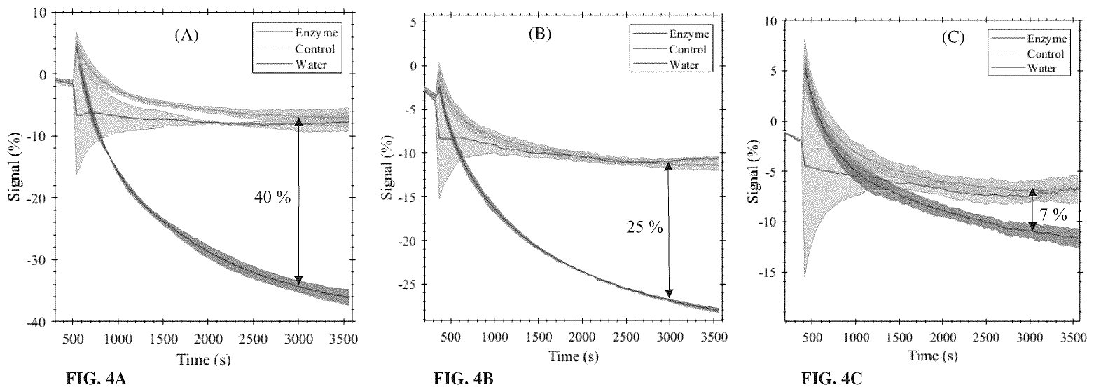

The results of enzyme activity screening for interrow, row and rhizosphere soil samples are shown in FIG. 4. To determine the difference in the enzyme activity of different soil samples, we labeled the difference between active enzymes and denatured controls as the difference index (D-index) at a set time point (3000 s). As can be seen, among the studied soil samples, the rhizosphere zone with a D-index of approximately 40% exhibited the highest level of activity compared to the row zone with 15% and the interrow zone with 7%, thereby confirming activity differences based on soil zone and rich literature on potential enzyme activity in relation to plant roots.

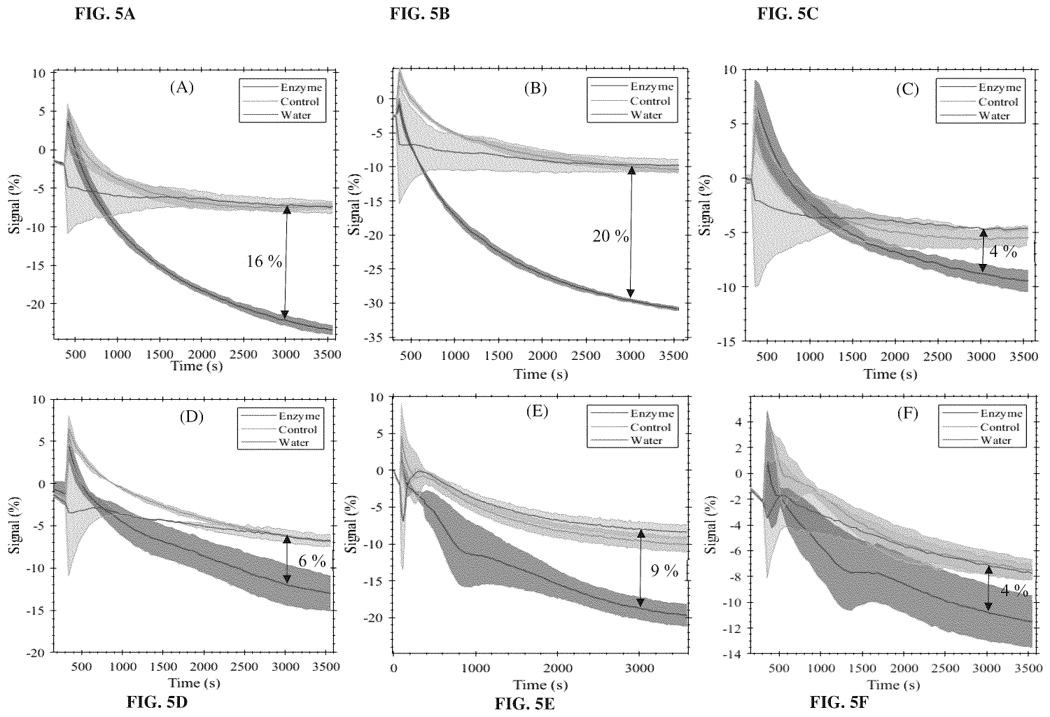

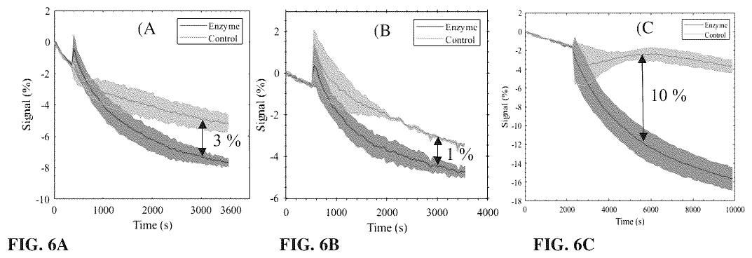

To observe the impact of storage condition and time on enzyme degradation, the enzyme screening was extended in the rhizosphere zone to monitor the gradual decline of the D-index. This was necessary because, in other areas, the enzyme activity was significantly lower, causing them to be degraded more rapidly and rendering conclusive observations impossible. The rhizosphere soil samples were kept under three storage temperatures-20, 4, and 25° C., and the enzyme activity screening analysis was performed after two and four weeks. Storing at 4° C. resulted in the least negative effect on the activity of enzymes available in soil samples compared to the storage at −20° C. and 25° C., however, all stored samples showed an attenuated signal to that measured from fresh soil, indicating the importance of fast screening or field side methods, as we have attempted with these nanoprobes. Degradation in storage has been attributed to instability of enzymes and aggregation as is common with other protein products. To assess the performance of LLS with a variety of soil textures, enzyme activity screening was conducted on fresh Nicollet soil and two soil types, Coland and Sparta, which had been stored for over four years at 25° C. and then had their enzyme activities reactivated. As illustrated in FIG. 6, LLS successfully detected active enzymes in all soil samples, with the largest change observed in the fresh Nicollet samples. It is thought that these air-dried samples may be biasing measurement of enzymes stabilized on clay particles, as the soils that are reading higher have This indicates one potential use of spatial mapping of enzyme activity caused by varied soils that differ largely in texture, soil organic matter, water holding capacity, and cation exchange capacity; not to mention their soil biological potential when substrates are added. As shown in FIG. 5, relative to fresh soil, and refrigerated and frozen, the air-dried soil potential enzyme activity was much lower (36% signal reduction).

### Modularity of Mechanochemical Approach

As another test of modularity of this method, a Zein-Lysine-SWCNT sensor was prepared using the same procedure as for the LLS. Zein is a water insoluble protein derived from corn. This sensor was then employed to evaluate the enzyme activity of bacterial protease. In this experiment, 50 mg of bacterial protease pellets (Carolina 202390) were dissolved in DI water. The resulting solution was then used along with Zein-Lysine-SWCNT sensor in a ratio of 20 μl Zein-Lysine-SWCNT to 80 μl bacterial protease solution, as detailed in nIR experiment section.

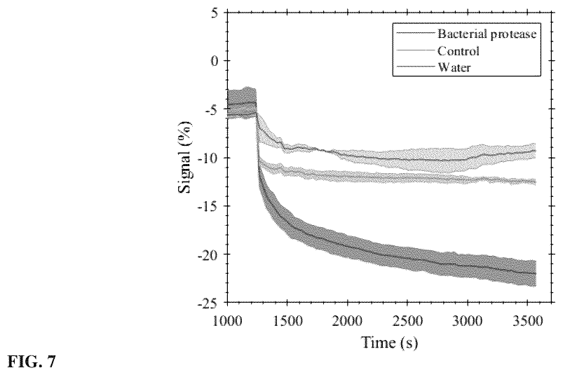

As illustrated in FIG. 7, the coupling process was successful, and the Zein-Lysine-SWCNT sensor effectively screened the enzyme activity in the bacterial protease solution vs. a heat denatured control.

This example, a facile mechanochemical strategy is proposed to render hydrophobic substrates suitable for non-covalent association to SWCNT for screening hydrolase activity in aqueous buffers. This was done by tip sonication of the target insoluble substrate in the presence of lysine, forming ion pairs between the lysine and carboxylic acid groups present on the substrate. The coupled substrates are then used to suspend SWCNT and form a probe for hydrolase activity measurements. As a focusing application, we coupled lignin to lysine and suspended SWCNT probes (LLS) and measured relative enzyme activity in varied root zones and storage conditions. LLS corroborated literature precedence, such as maximum activity in fresh samples close to the root. Another hydrophobic substrate, zein, was coupled with the same approach and made a sensor sensitive to bacterial protease. Furthermore, it is verified the chemistry of the coupling process by designing a similar coupling reaction between lysine and anthracenes with single carboxylic acid group, supporting our hypotheses regarding the N−COOH coupling to form ion pairs. The result of this enables a large library of natural, hydrophobic substrates to be rendered into soluble activity probes for high throughput screening applications such as discover and optimization of enzymes for valorization of biomass.

### Additional Aspects

The following exemplary aspects are provided, the numbering of which is not to be construed as designating levels of importance:

Aspect 1 provides a sensor assembly probe for determining enzymatic activity, the sensor assembly probe comprising:
a fluorescent semi-conductive nanoparticle;
a hydrophobic substrate for a predetermined enzyme, associated with the fluorescent semi-conductive nanoparticle by a non-covalent electrostatic interaction; and
a hydrophilic group bonded to the hydrophobic substrate.

Aspect 2 provides the sensor assembly probe of Aspect 1, wherein a morphology of the fluorescent semi-conductive nanoparticle comprises a nanosphere, a nanorod, a nanofiber, a nanotube, a nanostar, a nanocup, or combinations thereof.

Aspect 3 provides the sensor assembly probe of any of Aspects 1 or 2, wherein at least one of a length, width, and diameter of the fluorescent semi-conductive nanoparticle is in a range of from about 0.5 nm to about 100 nm.

Aspect 4 provides the sensor assembly probe of any of Aspects 1-3, wherein a particle size of the fluorescent semi-conductive nanoparticle is in a range of from about 10 nm to about 50 nm.

Aspect 5 provides the sensor assembly probe of any of Aspects 1-4, wherein the fluorescent semi-conductive nanoparticle comprises a ceramic, a polymer, a metal carbide, a nitride, a metal, graphite, carbon, or a mixture thereof.

Aspect 6 provides the sensor assembly probe of any of Aspects 1-5, wherein the fluorescent semi-conductive nanoparticle is a carbon nanotube.

Aspect 7 provides the sensor assembly probe of any of Aspects 1-6, wherein the fluorescent semi-conductive nanoparticle fluoresces at frequency ranging from about 800 nm to about 1500 nm.

Aspect 8 provides the sensor assembly probe of any of Aspects 1-7, wherein the fluorescent semi-conductive nanoparticle fluoresces at frequency ranging from about 950 nm to about 1100 nm.

Aspect 9 provides the sensor assembly probe of any of Aspects 1-8, wherein the hydrophobic substrate comprises a carboxylic acid.

Aspect 10 provides the sensor assembly probe of Aspect 9, wherein the hydrophobic substrate comprises lignin, a polysaccharide, a protein, or a lipid.

Aspect 11 provides the sensor assembly probe of any of Aspects 1-10, wherein the hydrophilic group comprises an amine group.

Aspect 12 provides the sensor assembly probe of Aspect 11, wherein the hydrophilic group is an amino acid.

Aspect 13 provides the sensor assembly probe of Aspect 12, wherein the amino acid is lysine.

Aspect 14 provides the sensor assembly probe of any of Aspects 9-13, wherein the hydrophilic group and the substrate are joined by an amide bond formed between the amine of the linker and the carboxylic acid of the substrate.

Aspect 15 provides the sensor assembly probe of any of Aspects 1-14, wherein the predetermined enzyme comprises a hydrolase, an oxidase, a cellulase, a protease or a mixture thereof.

Aspect 16 provides the sensor assembly probe of Aspect 15, wherein the hydrolase is chosen from an esterase, a nuclease, a phosphodiesterase, a lipase, a phosphatase, a DNA glycosylase, a glycoside hydrolase, a protease, a peptidase, an acid anhydride hydrolase, a helicase, a GTPase, or a mixture thereof.

Aspect 17 provides the sensor assembly probe of any of Aspects 15 or 16, wherein the protease comprises a cysteineprotease, a serineprotease, a threonineprotease, an aspartic protease, a glutamic protease, a metalloprotease, a PA clan protease, or a mixture thereof.

Aspect 18 provides the sensor assembly probe of any of Aspects 15-17, wherein the cellulase comprises endo-1,4-beta-D-glucanase (beta-1,4-glucanase, beta-1,4-endoglucan hydrolase, endoglucanase D, 1,4-(1,3,1,4)-beta-D-glucan 4-glucanohydrolase), carboxymethyl cellulase (CMCase), avicelase, celludextrinase, cellulase A, cellulosin AP, alkali cellulase, cellulase A 3, 9.5 cellulase, and pancellase SS.

Aspect 19 provides the sensor assembly probe of any of Aspects 15-18, wherein the oxidase comprises glucose oxidase, monoamine oxidase, cytochrome p450 oxidase, NADPH oxidase, xanthine oxidase, L-gulonolactone oxidase, laccase, lysyl oxidase, polyphenol oxidase, sulfhydryl oxidase, or a mixture thereof.

Aspect 20 provides the sensor assembly probe of any of Aspects 1-19, wherein the fluorescent semi-conductive nanoparticle is a first fluorescent semi-conductive nanoparticle and the assembly further comprises a second fluorescent semi-conductive nanoparticle.

Aspect 21 provides the sensor assembly probe of Aspect 20, wherein the first fluorescent semi-conductive nanoparticle and the second fluorescent semi-conductive nanoparticle have substantially the same composition.

Aspect 22 provides the sensor assembly probe of Aspect 20, wherein the first fluorescent semi-conductive nanoparticle and the second fluorescent semi-conductive nanoparticle have different compositions.

Aspect 23 provides the sensor assembly probe of any of Aspects 20-22, wherein the first fluorescent semi-conductive nanoparticle and the second fluorescent semi-conductive nanoparticle fluoresce at different frequencies.

Aspect 24 provides the sensor assembly probe of Aspect 23, wherein the respective fluorescent signals emitted by the first fluorescent semi-conductive nanoparticle and the second fluorescent semi-conductive nanoparticle have frequencies of fluorescence that differ by about 0% to about 100%, relative to each other.

Aspect 25 provides the sensor assembly probe of any of Aspects 23 or 24, wherein the respective fluorescent signals emitted by the first fluorescent semi-conductive nanoparticle and the second fluorescent semi-conductive nanoparticle have frequencies of fluorescence that differ by about 0% to about 20%, relative to each other.

Aspect 26 provides a sensor assembly comprising the probe of any of Aspects 1-25, the sensor assembly further comprising the predetermined enzyme.

Aspect 27 provides the sensor assembly of Aspect 26, wherein the predetermined enzyme is a first enzyme and the assembly further comprises a second enzyme.

Aspect 28 provides a method of using the sensor assembly probe of any of Aspects 1-27, the method comprising:
measuring a first fluorescent frequency emission of the probe;
contacting the substrate and the predetermined enzyme; and
measuring a second fluorescent frequency emission of the probe, wherein the second fluorescent frequency emission is less than the first fluorescent frequency emission and indicates that at least a portion the substrate has reacted with the predetermined enzyme.

Aspect 29 provides the method of Aspect 28, wherein the second fluorescent frequency emission is zero.

Aspect 30 provides the method of any of Aspects 28 or 29, wherein a mixture of enzymes comprises the predetermined enzyme.

Aspect 31 provides the method of any of Aspects 28-30, further comprising determining a rate of reaction between the substrate and the predetermined enzyme.

Aspect 32 provides the method of Aspects 31, wherein determining a rate of reaction comprises measuring a plurality of fluorescent signals over a predetermined amount of time to quantify the amount of substrate that is consumed by the predetermined enzyme.

Aspect 33 provides a method of making the sensor assembly probe of any of Aspects 1-32, the method comprising:
contacting the hydrophobic substrate and the hydrophilic group;
exposing the hydrophobic substrate and the hydrophilic group to a first sonication step to bond the hydrophobic substrate and the hydrophilic group;
washing away any non-bonded hydrophilic group;
contacting the bonded hydrophobic substrate and the hydrophilic group with the semi-conductive nanoparticle;
exposing the bonded hydrophobic substrate and hydrophilic group and the semi-conductive nanoparticle to a second sonication step to form the non-covalent electrostatic interaction and form the sensor assembly.

Aspect 34 provides the method of Aspect 33, wherein the first sonication step and second sonication step independently occurs for a time in a range of from about 5 minutes to about 60 minutes.

Aspect 35 provides the method of any of Aspects 33 or 34, wherein the first sonication step and second sonication step independently occurs for a time in a range of from about 15 minutes to about 25 minutes.

What is claimed is:

1. A sensor assembly probe for determining enzymatic activity, the sensor assembly probe comprising:
a fluorescent semi-conductive nanoparticle;
a hydrophobic substrate for a predetermined enzyme, associated with the fluorescent semi-conductive nanoparticle by a non-covalent electrostatic interaction; and
a hydrophilic group bonded to the hydrophobic substrate.

2. The sensor assembly probe of claim 1, wherein a morphology of the fluorescent semi-conductive nanoparticle comprises a nanosphere, a nanorod, a nanofiber, a nanotube, a nanostar, a nanocup, or combinations thereof.

3. The sensor assembly probe of claim 1, wherein at least one of a length, width, and diameter of the fluorescent semi-conductive nanoparticle is in a range of from about 0.5 nm to about 100 nm.

4. The sensor assembly probe of claim 1, wherein the fluorescent semi-conductive nanoparticle comprises a ceramic, a polymer, a metal carbide, a nitride, a metal, graphite, carbon, or a mixture thereof.

5. The sensor assembly probe of claim 1, wherein the fluorescent semi-conductive nanoparticle is a carbon nanotube.

6. The sensor assembly probe of any of claim 1, wherein the fluorescent semi-conductive nanoparticle fluoresces at frequency ranging from about 800 nm to about 1500 nm.

7. The sensor assembly probe of claim 1, wherein the hydrophobic substrate comprises a carboxylic acid.

8. The sensor assembly probe of claim 7, wherein the hydrophobic substrate comprises lignin, a polysaccharide, a protein, or a lipid.

9. The sensor assembly probe of claim 1, wherein the hydrophilic group comprises an amine group.

10. The sensor assembly probe of claim 9, wherein the hydrophilic group and the substrate are joined by an amide bond formed between the amine of the hydrophilic group and the carboxylic acid of the substrate.

11. The sensor assembly probe of claim 1, wherein the predetermined enzyme comprises a hydrolase, an oxidase, a cellulase, a protease or a mixture thereof.

12. The sensor assembly probe of claim 11, wherein:
the hydrolase is chosen from an esterase, a nuclease, a phosphodiesterase, a lipase, a phosphatase, a DNA glycosylase, a glycoside hydrolase, a protease, a peptidase, an acid anhydride hydrolase, a helicase, a GTPase, or a mixture thereof;
the protease comprises a cysteineprotease, a serineprotease, a threonineprotease, an aspartic protease, a glutamic protease, a metalloprotease, a PA clan protease, or a mixture thereof;
the cellulase comprises endo-1,4-beta-D-glucanase (beta-1,4-glucanase, beta-1,4-endoglucan hydrolase, endoglucanase D, 1,4-(1,3,1,4)-beta-D-glucan 4-glucanohydrolase), carboxymethyl cellulase (CMCase), avicelase, celludextrinase, cellulase A, cellulosin AP, alkali cellulase, cellulase A 3, 9.5 cellulase, and pancellase SS; or
the oxidase comprises glucose oxidase, monoamine oxidase, cytochrome p450 oxidase, NADPH oxidase, xanthine oxidase, L-gulonolactone oxidase, laccase, lysyl oxidase, polyphenol oxidase, sulfhydryl oxidase, or a mixture thereof.

13. The sensor assembly probe of claim 1, wherein the fluorescent semi-conductive nanoparticle is a first fluorescent semi-conductive nanoparticle and the assembly further comprises a second fluorescent semi-conductive nanoparticle.

14. The sensor assembly probe of claim 13, wherein the first fluorescent semi-conductive nanoparticle and the second fluorescent semi-conductive nanoparticle fluoresce at different frequencies.

15. A method of using the sensor assembly probe of claim 1, the method comprising:
measuring a first fluorescent frequency emission of the probe;
contacting the substrate and the predetermined enzyme; and
measuring a second fluorescent frequency emission of the probe, wherein the second fluorescent frequency emission is less than the first fluorescent frequency emission and indicates that at least a portion the substrate has reacted with the predetermined enzyme.

16. The method of claim 15, wherein the second fluorescent frequency emission is zero.

17. The method of claim 15, wherein a mixture of enzymes comprises the predetermined enzyme.

18. The method of claim 15, further comprising determining a rate of reaction between the substrate and the predetermined enzyme.

19. A method of making the sensor assembly probe of claim 1, the method comprising:
contacting the hydrophobic substrate and the hydrophilic group;
exposing the hydrophobic substrate and the hydrophilic group to a first sonication step to bond the hydrophobic substrate and the hydrophilic group;
washing away any non-bonded hydrophilic group;
contacting the bonded hydrophobic substrate and the hydrophilic group with the semi-conductive nanoparticle;
exposing the bonded hydrophobic substrate and hydrophilic group and the semi-conductive nanoparticle to a second sonication step to form the non-covalent electrostatic interaction and form the sensor assembly.

20. The method of claim 19, wherein the first sonication step and second sonication step independently occurs for a time in a range of from about 5 minutes to about 60 minutes.
